(19) United States
(12) Patent Application Publication
(10) Pub. No.: US 2002/0037558 A1
(43) Pub. Date: Mar. 28, 2002

(54) E.COLI PRODUCED IMMUNOGLOBULIN CONSTRUCTS

(76) Inventors: KIN-MING LO, WELLESLEY, MA (US); STEPHEN D. GILLIES, HINGHAM, MA (US)

Correspondence Address: TESTA, HURWITZ & THIBEAULT, LLP, HIGH STREET TOWER, 125 HIGH STREET, BOSTON, MA, 02110, US

(21) Appl. No.: 07/781,395

(22) Filed: Oct. 23, 1991

(51) Int. Cl. C12Q 1/68; C12P 21/06; C12P 21/04; A61K 39/395; C12N 1/00; C12N 1/20; C07K 16/00

(52) U.S. Cl. 435/69.6; 435/243; 435/252.8; 424/130.1; 530/387.1

(57) ABSTRACT

A method of producing recombinant heterotetrameric immunoglobulin from a procaryotic organism which includes providing a procaryotic organism that has been transformed with DNA encoding the heavy and light chains of an immunoglobulin having a binding site for immunologically binding a preselected antigen and an amino acid sequence which signals the export of the immunoglobulin from the cytoplasm of the organism, the DNA being operationally associated with a promoter recognizable by RNA polymerase endogenous to the organism, and culturing the transformed procaryote for a time and under conditions sufficient to allow the organism to export the immunoglobulin from the cytoplasm, wherein the exported heterotetrameric immunoglobulin retains its native conformation and binding specificity for the preselected antigen.

### The invention relates to obtaining immunoglobulin molecules from a procaryotic microorganism.

### BACKGROUND OF THE INVENTION

E. coli is widely used for the production of recombinant proteins, but the bacterial expression and secretion of an assembled, complex, heterotetrameric mammalian antibody molecule has not been successful. Problems encountered in the bacterial expression of heterologous proteins include the reducing nature of the intracellular environment, the insolubility of the recombinant proteins, and what appears to be a lack of assembly apparatus in the cytoplasm of E. coli (Mitraki et al., 1989, Bio/Technology 7:690-697). Cabilly et al. (1984, Proc. Natl. Acad. Sci, USA 81:3273-3277) and Boss et al. (1984, Nucleic Acids Res. 12:3791-3806) co-expressed the light (L) and heavy (H) chain genes of the immunoglobulin molecule in E. coli, and obtained both proteins as insoluble products in the form of inclusion bodies. No detectable antibody activity was found in the cell lysates, and antigen-binding activity could be obtained only by in vitro solubilization of the inclusion bodies, followed by renaturation. The yield of reconstituted antibody is frequently low.

The use of a signal peptide derived from an outer membrane protein, e.g., ompA, reportedly allows export of the dimeric Fv fragment into the periplasmic space of E. coli (Skerra et al., 1988, Science 240: 1038-1041), and allows secretion of the dimeric Fab fragment into the culture medium (Better et al., 1988, Science 240: 10411043). FIG. 1 illustrates an antibody molecule and the sites of cleavage of the molecule to generate Fv and Fab dimeric fragments. Pluckthun (1991, Bio/Technology 9:545551) notes that Fab molecules can be produced in E. coli, but that the production and folding of whole antibodies in E. coli “may have folding or stability deficiencies in the Fc part” of the molecule.

The mouse/human chimeric antibody, ch14.18, made by conventional technology. (See Gillies et al, 1989 J. Immunol. Methods, 125: 191-202.) reacts with the disialoganglioside GD2 on the surface of tumor cells of neuroectodermal origin with enhanced antibody-dependent cytotoxicity (Mueller et al., 1990, J. Immunol. 144: 1382). The CH2 deletion variant immunoglobulin, ch14.18ΔCH2, which includes only the CH1, hinge, and CH3 domains of the constant region of the antibody molecule, has been shown to be a potentially useful reagent for radioimmunodetection of human tumors because of its reduced immunogenicity, increased target specificity, and rapid clearance from circulation (Mueller et al., 1990, Proc. Natl Acad. Sci. USA 87:5702-5705).

It is an object of the invention to facilitate the production of assembled heterotetrameric immunoglobulin without extensive purification and without denaturation and renaturation of the immunoglobulin molecule. Another object of the invention is to produce such immunoglobulin as a protein product which is exported from bacteria into the periplasmic space or further secreted into the culture medium.

### SUMMARY OF THE INVENTION

The invention is based on the discovery that a procaryotic organism can be engineered to export out of the cytoplasm a fully assembled foreign heterotetrameric antibody molecule that retains its native conformation upon export and is able to immunologically bind a preselected antigen.

As used herein, a “heterotetrameric” antibody or immunoglobulin includes a four-chain antibody molecule, i.e., that contains two pairs of polypeptide chains: two heavy chains and two light chains. The “light” chain of an antibody includes the full-length variable and constant regions, whereas the “heavy” chain includes either the full-length variable and constant regions, or the full-length variable region and less than the full-length constant region, but enough of the constant region to allow the molecule to form a heterotetramer, e.g., a CH2-deleted immunoglobulin, or an immunoglobulin molecule without deletions or truncations. Preferred binding proteins made in accordance with the invention include the CH3 domain. “Heterotetrameric” antibody or immunoglobulin also include fusion proteins comprising an independently biologically functional polypeptide bonded to the C-terminus of the CH3 domain, e.g., a lymphokine, cytokine, or cell toxin. The “native conformation” of an antibody means the conformation that in all material respects mimics the tertiary structure taken by an antibody that is produced by B cells in the human body, or by antibody-producing cells, e.g., hybridoma or myeloma cells, in culture; and “immunological binding” refers to the noncovalent interactions that occur between an antibody and its cognate antigen.

In its broadest aspects, the invention features a method for producing an antibody in its native conformation using a procaryotic organism as a host cell, and the antibody or antibody fusion constructs produced by that method. The method includes providing a procaryotic organism that has been transformed with DNA encoding the heavy and light chains of an immunoglobulin having a binding site for immunologically binding a preselected antigen and an amino acid sequence which signals the export of the immunoglobulin from the cytoplasm of the organism, wherein the transforming DNA is operationally associated with a promoter recognizable by RNA polymerase endogenous to the organism, and culturing the transformed procaryote for a time and under conditions sufficient to allow the organism to export the immunoglobulin from the cytoplasm of the organism, e.g., into the periplasmic space and/or into the culture medium surrounding the cultured organism. The exported immunoglobulin is a fully assembled heterotetrameric protein that retains its native conformation and its binding specificity for the preselected antigen.

A used herein, a promoter is “operationally associated” with DNA encoding a protein when it is arranged so as to promote transcription of the coding DNA; a promoter that is “recognizable” by an endogenous RNA polymerase is any promoter-specifying sequence that promotes transcription by an RNA polymerase; an “endogenous” RNA polymerase is one which is present in the organism either naturally or by design, i.e., that which is naturally found in the untransformed procaryotic organism, or is introduced into the host organism by recombinant DNA techniques. An “export” sequence refers to an amino acid sequence, fused to a protein, that directs the host cell to export the protein out of the cytoplasm into the periplasmic space and/or into the surrounding culture medium.

In another embodiment, the invention features a recombinant DNA encoding a heterotetrameric immunoglobulin, which includes a heavy chain and a light chain, and an amino acid sequence which directs export of the immunoglobulin from the organism's cytoplasm into either the periplasm or both the periplasm and the culture medium, the DNA being operationally associated with a promoter recognizable by RNA polymerase endogenous to the organism, whereby, upon expression in the host, there is exported an immunoglobulin construct including a binding site for a preselected antigen in its native antigen-binding conformation. The DNA may also encode, 3′ of the region encoding the immunoglobulin domain, another single chain polypeptide having a conformation which confers the native biological activity to the polypeptide.

In preferred embodiments, the procaryotic organism is a gram negative bacterium; preferably E. coli. In other preferred embodiments, the export sequence preferably is a bacterial export sequence, e.g., one of the E. coli pectate lyase (pel) B, ompA, phoA, ompF, or alkaline phosphatase signal sequences (other useful signal sequences include but are not limited to those derived from secretory proteins of bacterial or mammalian origins). Any sequence which directs transport across the inner membrane may be used; preferably, the export sequence is pelB and the immunoglobulin is exported into the culture medium surrounding the cultured organism. In preferred embodiments, the heavy chain encoding DNA may include the complete H chain amino acid sequence or may contain a deletion of DNA encoding the immunoglobulin CH2 domain; most preferably, the CH2 deletion is identical to that which encodes the ch14.18ΔCH2 antibody.

One advantage of the method of the invention is that it is a relatively fast and easy way of obtaining heterotetrameric immunoglobulin without extensive purification and without denaturation and renaturation of the immunoglobulin molecule. Recombinant heterotetrameric immunoglobulins of the invention retain the native conformation of the immunoglobulin molecule and are able to bind a preselected antigen with the same affinity as antibodies obtained naturally or produced by mammalian cells. These immunoglobulins are useful as reagents in techniques where antigen binding is required, e.g., in immunotherapy or immunodiagnosis, as catalytic antibodies, or in screening of combinatorial library of antibody repertoire in E. coli (Huse et al., 1989, Science 246:1275-1281).

Other advantages of the invention include the following: The bacterial production of antibody facilitates the production of immunotoxins by genetic engineering, because the toxin moiety is often extremely toxic to the mammalian host, but not to bacteria. Accumulation of antibody in a cell culture medium rather than in the bacterial cytoplasm significantly reduces the number of contaminating bacterial proteins and the potential degradation problem caused by bacterial proteases. Secreted protein, with the signal peptide correctly processed, has the correctly processed amino terminus, i.e., without the fMet, which is the initiation codon in bacteria. Production of immunoglobulin in its native conformation not only renders itself amenable to purification by affinity chromatography, including binding to immobilized antigen, but also renders it more resistant to protease degradation due to its correctly folded globular domains. Obtaining divalent antibody from E. coli is more advantageous than, e.g., monovalent Fab fragment, due to the greater affinity in antigen binding of the divalent antibody. In cases where the antigen is polymeric or bound on the surface, and when the thermodynamic affinity of a single binding site is relatively weak, divalent heterotetrameric antibody is useful to generate a high avidity antibody for detection of antigen.

### BRIEF DESCRIPTION OF THE DRAWINGS

FIG. 1 illustrates an antibody molecule and the sites of cleavage to generate Fv and Fab fragments.

FIG. 2 is a map of the bacterial expression vector pKKx-pelB 14.18ΔCH2.

FIGS. 3A and B are Coomassie blue staining of polyacrylamide gel analysis of ch14.18ΔCH2 antibody purified from minimal culture media of E. coli.

FIG. 4 is an elution profile of bacterial produced ch14.18ΔCH2 antibody using non-denaturing size exclusion high pressure liquid chromatography.

FIGS. 5A and B are graphs of antigen binding assays of a bacterial produced ch14.18 ΔCH2 antibody using GD2coated plates.

FIGS. 6A-C and 7A-C show electrophoretic analysis under reducing and non-reducing conditions, respectively, and subsequent immunoblotting of the ch14.18 antibody purified from E. coli Sp2/0 culture media.

FIG. 8 is an elution profile of bacterial-made ch14.18 antibody using non-denaturing size exclusion high pressure liquid chromatography.

FIG. 9 is a graph of competitive binding assay of a bacterial-made ch14.18 antibody using GD2-coated plates.

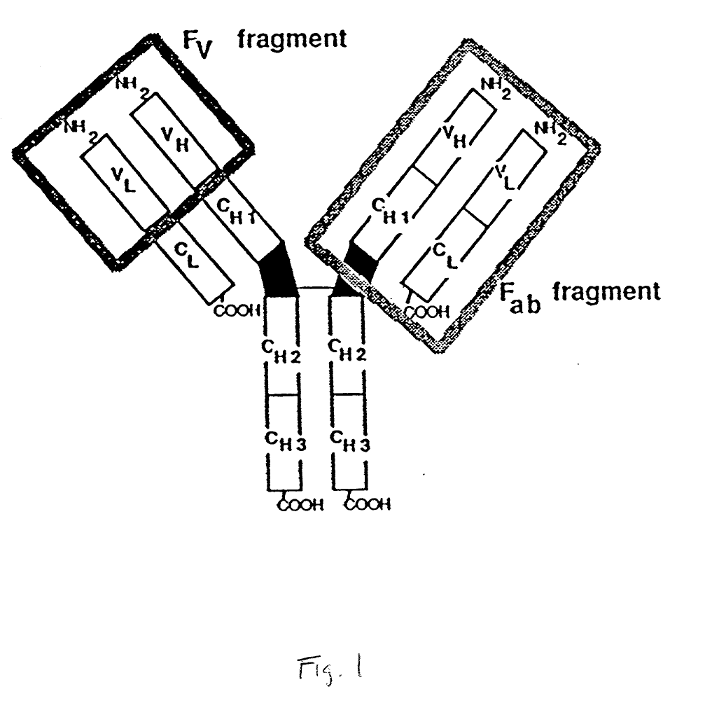

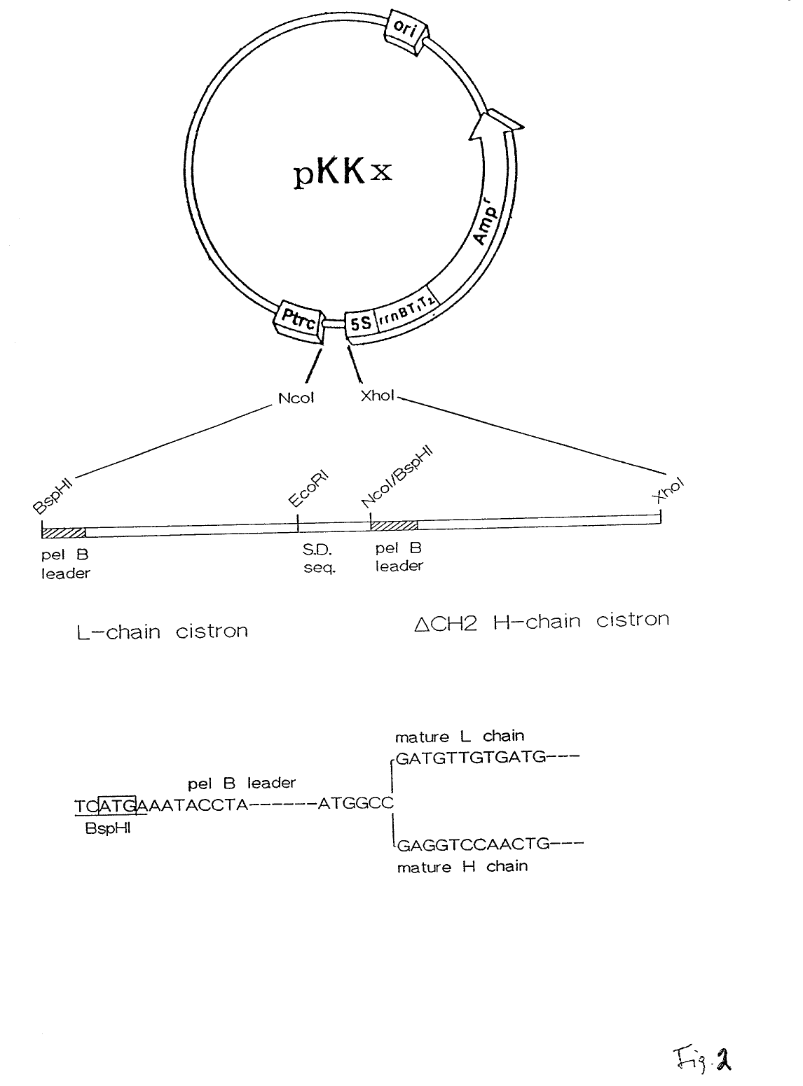

### DESCRIPTION OF EMBODIMENTS OF THE INVENTION

Procaryotic, e.g., bacterial-produced antibodies of the invention may be expressed as functional, fully-assembled heterotetrameric antibody that is exported from the bacterial cytoplasm and into the periplasm or culture medium. Any immunoglobulin isotype may be produced from a procaryotic organism according to the invention, as may any truncated immunoglobulin molecule that is capable of forming an H2:L2 heterotetramer. Procaryotic organisms useful as transformed hosts capable of producing antibodies include but are not limited to gram negative bacteria, e.g., E. coli or Bacillus subtilis, or gram positive bacteria.

In order to produce antibody from a procaryotic organism according to the invention, it is preferable to select an export sequence and engineer the gene encoding the immunoglobulin so as to replace the signal sequence which naturally occurs at the 5′ end of the H or L chain coding sequence with DNA encoding an export sequence that is recognized by the export assembly of the organism. The bacterial export sequence will be produced as part of a fusion protein and will be fused to each of the amino termini of the H and L chains. The export sequence will thus direct export of the immunoglobulin, in assembled or nonassembled form, out of the bacterial cytoplasm and into the periplasm and/or culture medium, where the immunoglobulin appears in a fully assembled native conformation. During export, the export sequence is cleaved off to yield the mature protein, with a correctly processed amino terminus. An example of a preferred export sequence for export of an immunoglobulin from the cytoplasm of E. coli is the E. coli pectate lyase B signal sequence (Lei et al., 1987, J. Bacteriol. 169:4379-4383). Once the H and L chains are exported out of cytoplasm, they are found in the native assembled heterotetrameric conformation of an immunoglobulin, and thus do not need to be denatured and renatured.

A preferred embodiment of the invention is the production of a CH2-deleted chimeric antibody, ch14.18ΔCH2. This antibody lacks the CH2 domain, which contains many of the effector functions and the sole N-linked glycosylation site in human Cγ1. Experiments described below in Examples 1-13, disclose how to carry out the invention. Example 1 describes the construction of a bacterial expression vector encoding immunoglobulin H and L chains. The vector contains DNA encoding a dicistronic unit including a L-chain cDNA and a CH2-deleted H-chain CDNA. Example 2 describes expression of the dicistronic unit in a JM105 E. coli host, using a regulatory region which includes the E. coli trc promoter. Examples 3 and 4 demonstrate translocation of the immunoglobulin across the bacterial membranes using the pectate lyase B (pelB) signal peptide in place of the natural signal peptides of the H and L chains, and quantitation of the immunoglobulin product secreted into the M9 growth media. The secreted antibody, which can be readily purified from the media without any denaturation of renaturation steps, retains antigen-binding activity, as described in Example 5. The results of SDS-PAGE and nondenaturing high pressure exclusion chromatography, described in Example 4, show that the E. coli-produced immunoglobulin is a mixture of assembled HL heterodimer and fully assembled H2L2 heterotetramer. Examples 7-12 further illustrate the invention using a complete H chain, i.e., without deletion of the CH2 domain.

Analysis of the culture media and cell lysates demonstrated that 80% to 90% of the ch14.18ΔCH2 antibody accumulated in the media. The use of minimal media, which introduces no extraneous protein to the culture, further facilitates the concentration and purification steps. The observation that functional tetrameric antibody can be recovered from the bacterial culture medium obviates the need for any in vitro renaturation and makes this expression system very attractive.

1. Construction of Bacterial Expression Vector pKKx-pelB 14.18ΔCH2.

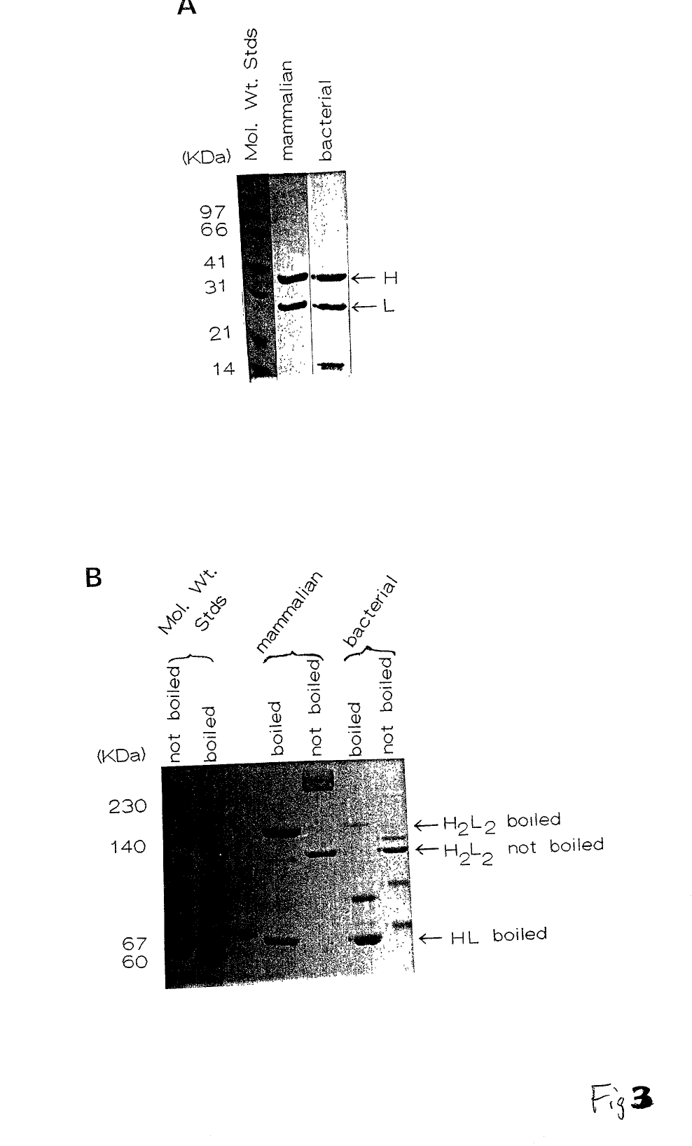

The expression vector pKKx-pelB 14.18ΔCH2, shown in FIG. 2, was derived from pKK233-2 (Pharmacia, Piscataway, N.J.). In FIG. 2, “Ptrc” indicates the trc promoter; “Amp^{r}”, the ampicillin resistance gene; “ori”, the replication origin of pBR322; and “S.D. sequence”, the Shine-Dalgarno sequence. The DNA sequences of the pelB export sequence fused to the mature L and H chain junctions are given. The boxed ATG is the translation initiation codon. pKK233-2 was first linearized with HindIII, and the single stranded ends filled in with Klenow and dNTP's and ligated to an XhoI linker (New England Biolabs, Beverly, Mass.). The resultant construct was cloned and designated pKKx, i.e., pKK233-2 with an XhoI site.

pKKx-pelB 14.18ΔCH2 contains a dicistronic operon under the control of the trc promoter. The trc promoter includes a consensus 17 bp spacing between the trp −35 region and lacUV5 −10 region (de Boer et al., 1983, Proc. Natl. Acad. Sci. USA 80:21-25). The dicistronic operon, cloned into the unique NcoI and XhoI sites of pKKx (FIG. 2), was constructed as follows.

Poly A⁺ enriched mRNA was prepared from a transfected Sp2/0 cell line which produces the ch14.18ΔCH2 antibody (Gillies et al., 1990, Hum. Antibod. Hybridomas 1:47-54). First strand cDNA synthesis was performed as described by Gubler et al. (1983, Gene 25:263-269) and approximately 1 pg of the cDNA-mRNA hybrid was used as template for polymerase chain reaction (PCR). All PCRs were performed using the GeneAmp DNA amplification reagent kit (Perkin-Elmer/Cetus, Norwalk, Conn.) in a Perkin Elmer/Cetus Thermal Cycler, following the reaction conditions recommended by the supplier. The PCR products containing the coding regions of the L and H chains were cloned separately into a Bluescrybe vector (Stratagene, LaJolla, Calif.). After DNA sequencing, the correct clones were used for further reconstruction.

The cloned cDNAs of the L and H chains already contain an EcoRI site and an XhoI site respectively at the 3′-end, through incorporation of these sites in the PCR primers. The 5′-ends were reconstructed to replace the natural leader peptides with that of pelB (Lei et al., 1987, supra). The BglII site at Arg-24 of the L chain and the PstI site at Leu-5 of the H chain, according to the amino acid numbering designation of +1 for the first amino acid at the N-terminus of the mature protein, were chosen for this purpose. Overlapping oligonucleotides encoding the initiation codon, the pel B signal peptide, and the N-termini of the mature L and H chains were synthesized and ligated (Lo et al., 1984, Proc. Natl. Acad. Sci. USA 81: 2285-2289). The resulting BspHI-BglII oligonucleotide duplex for the L chain and BspHI-PstI oligonucleotide duplex for the H chain were then joined to the rest of the mature sequences (BglII-EcoRI fragment of the L chain and PstI-XhoI fragment of the H chain) to form the two complete cistrons.

The complete L-chain cistron consists of the translation initiation codon, the pelB leader sequence followed immediately by the sequence of the mature L chain, and a translation termination codon. This cistron was constructed as a BspHI-EcoRI fragment so that, upon ligation of the compatible 5′ single stranded ends of the pelB BspHI site and the pKKx NcoI site, the ATGAAA sequence of the initiation codon and the first codon (lysine) of the pelB signal peptide is preserved (FIG. 2). The H-chain cistron was constructed similarly as a BspHI-XhoI fragment. Between the two cistrons is a 38 bp synthetic EcoRI-NcoI fragment containing the Shine-Delgarno (ribosome binding) sequence of the lacZ gene (corresponding to nucleotides 4315-4352 of pKK233-2). Hence, each cistron is preceded by a ribosome binding site to ensure efficient translation.

2. Cell growth, expression and induction.

The expression of pKKx-pelB14.18ΔCH2 was carried out in JM105, a lac host. Fresh colonies were scraped off from an LB-amp plate and seeded into 250 mL of LB-amp (50 μg/mL). At OD₅₅₀ of 0.6 to 1, the cells were pelleted and then resuspended in 1 L of M9-amp. After shaking at 37° C. overnight, the M9 culture medium was harvested by centrifugation at 5000 rpm for 30 min. In induction experiments, cells growing in LB-amp or M9-amp to OD₅₅₀ of about 0.8 were induced with 0.5, 1 or 5 mM of IPTG for 6 hrs. or overnight. Expression was monitored by assaying for antibodies in the media and plating titers of cells growing at different stages on LB and LB-amp plates.

The ch14.18ΔCH2 antibody expressed is sufficiently toxic to the bacterial host to prevent the establishment of the expression vector in JM11, which does not overproduce laci. Even in a lacI^{q} host such as JM105, proper care should be taken to ensure plasmid stability. Titers of cultures at various stages were plated on LB agar with ampicillin, IPTG, neither, or both added to determine the fraction of cells that retains the plasmid and the ability to express the gene of interest. The fraction of cells retaining the expressible plasmid should be over 98% (see Studier et al., 1977, Methods Enzymol. 185:60-89). Only fresh colonies from LB-amp plates were used for inoculation. In the stepwise scale-up of a large culture, the subculture was grown only to mid-log before the cells were collected by centrifugation. The subculture medium, which contained a large amount of β-lactamase, was discarded. The cell pellet was used as a heavy inoculum for the final culture, which was allowed to grow overnight. Use of the pelB leader peptide for secretion results in the secretion of products, which continues for at least several hours after inoculation and gives maximal accumulation after an overnight culture.

The trc promoter was not fully repressed even in a lacI^{q} host (de Boer et al., 1982, In: Promoter Structure and Function, eds. R. L. Rodriguez and M. J. Chamberlain, Praegar Publishers, New York), since a basal level of about 350 ng/mL of product was expressed and secreted into the medium.

When the culture was induced at mid-log with IPTG, however, little product accumulated after 6 hr, and the expression level of the culture induced overnight was about the same as that of the uninduced culture. Plating on LB and LB-amp plates revealed that after induction overnight, the culture was probably overgrown by cells lacking the plasmid. This was shown to be the case because the yield of plasmid DNA that could be prepared from the induced cells dropped drastically, though the culture still reached high optical density.

If the expression level in a bacterial antibody production system of the invention is moderate, the system may be optimized by varying several parameters; e.g., inducing gene expression using IPTG or another inducer/repressor system; replacing glucose with glycerol in the M9 media during induction, although if the lacUV5 promoter is used, there should be no catabolite repression; and/or translation optimization by taking into account codon usage in bacteria. Strategies to improve the secretion capability of the bacterial host include the cloning and expression in the same host of the kil gene (Kato et al., 1987, Gene 54:197-202) or the gene for the bacteriocin release protein (Hsiung et al., 1989, Bio/Technology 7:267), both of which are hereby incorporated by reference. The kil gene product leads to permeabilization of the outer membrane and subsequent release of the periplasmic proteins, and coexpression of bacteriocin release protein can lead to leakage of the protein of interest into the culture medium.

3. Electrophoretic Analysis of ch14.18ΔCH2 Antibody from E. coli.

For ease of purification, M9-amp media was used without IPTG induction for the final overnight culture during production. 6 L of the clarified M9 culture medium was filtered through 0.45 μm filters to remove any residual bacterial debris. Sodium azide was added to a final concentration of 0.02% and sodium hydroxide added to pH 7.0. The filtrate was then concentrated about ten-fold on a Minitan ultrafiltration system (Millipore) using a membrane with a 30 KDa cutoff. A 5-ml murine anti-human kappa Sepharose 4B column (Gillies et al., 1990, Hum. Antibod. Hybridomas 1:47-54) (capacity: 1.6 mg/mL) was equilibrated in PBS, pH 7.0. The sample was loaded at 50 mL/hr at 4° C. The column was washed first with PBS, pH 7.0, followed by a wash buffer containing 10 mM sodium phosphate and 500 mM NaCl, pH 7.0. The column was then eluted with PBS, pH 3.0. The peak fractions, as monitored by UV absorbance at 280 nm, were titrated to pH 7.0 and further concentrated in an Amicon stirred cell with a Diaflo ultrafiltration membrane YM5 to 0.34 mg/mL, as determined by anti(Fc) ELISA.

The expression level of antibody was slightly higher in the minimal media than in LB. Values obtained with the anti(H+L) ELISA were about one-third higher than those obtained with the anti(Fc) (an ELISA specific for the human Fc), indicating that there is free L chain or L chain dimer secreted into the media.

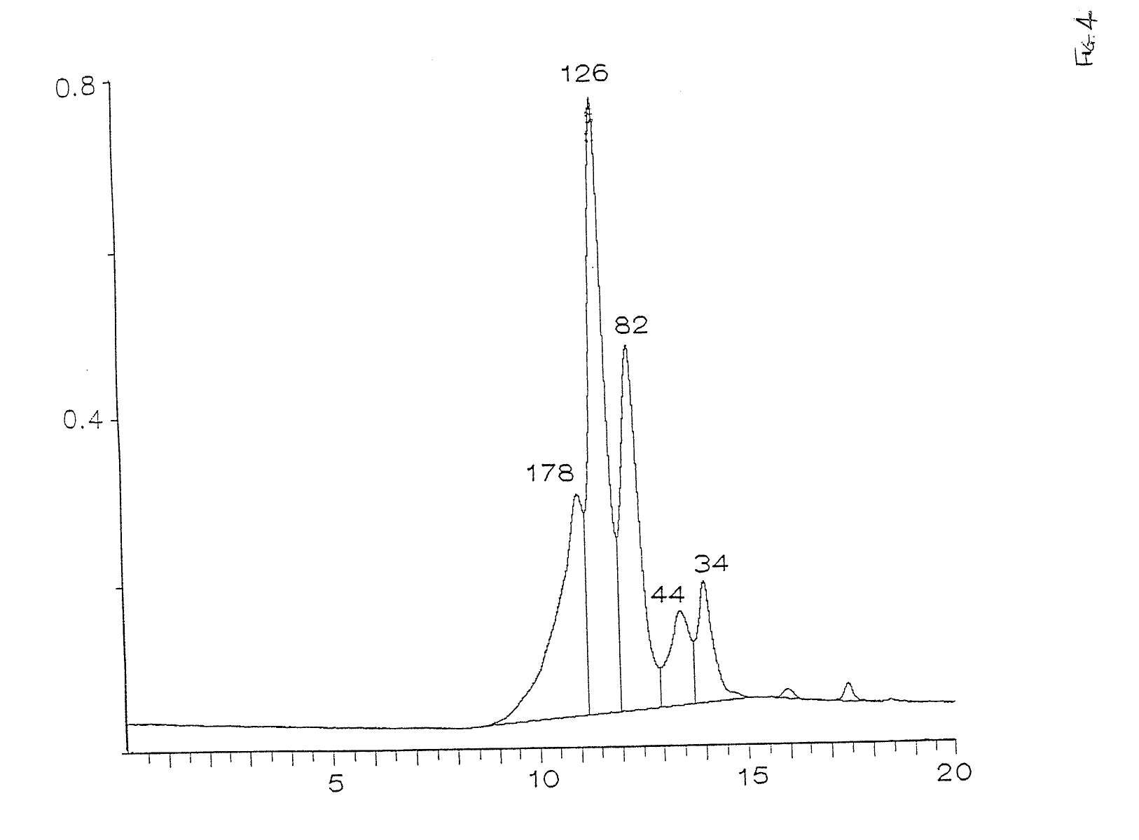

FIG. 3 shows results of polyacrylamide gel analysis of ch14.18ΔCH2 antibody purified from minimal culture media of E.coli. The ch14.18ΔCH2 antibody was purified on an anti-human K monoclonal antibody-Sepharose column and then further concentrated to 0.34 mg/mL, as determined by anti(Fc) ELISA. When analyzed by SDS-PAGE under reducing conditions, the H and L chains of the bacterial product were indistinguishable from their mammalian counterparts (see below and FIG. 3). When run under non-reducing conditions, there was a predominant species in the non-boiled bacterial product that corresponds to the mammalian H2L2. When the bacterial H2L2 was boiled, however, it gave rise to the HL half-molecule (FIG. 3B). This suggested that in the ch14.18ΔCH2 from E. coli, disulphide bonds are formed between the H and L chains but not between the two H chains. The two half molecules (HL) presumably are held together by the trans interaction between adjacent CH3 domains. The lack of inter-H chain disulphide bond formation can be partly due to the conformation of the ACH2 molecule, since about 40% of the ch14.18ΔCH2 antibody produced in mammalian cells also lack the inter-H chain disulphide bonds (see lane with the boiled sample of the mammalian preparation, FIG. 3B).

In FIG. 3, ch14.18ΔCH2 purified from spent culture of transfected Sp2/0 cells was used for comparison with the E. coli-produced antibody. In FIG. 3A, samples were analyzed on a 10% SDS-polyacrylamide gel after reduction with 2mercaptoethanol. The positions of the H and L chains are as indicated. The band at 14 KDa is a bacterial protein unrelated to immunoglobulin. In FIG. 3B, samples, boiled or not boiled, were run on a 7% SDS polyacrylamide gel under non-reducing conditions. H and L chain compositions of the species are indicated. H2L2 represents the full tetrameric antibody and HL is the half-molecule. The ch14.18ΔCH2 antibody from the Sp2/0 cells tends to aggregate slowly over time. The high mol. wt. band in the “not boiled” lane of the mammalian preparation is probably a dimer of the ch14.18ΔCH2 antibody.

4. Analysis of Antibody by Immunoblotting, Protein Sequencing and HPLC.

The identities of the bands assigned H2L2, HL (nonreducing gel, FIG. 3B), H and L (reducing gel, FIG. 3A) were further confirmed by immunoblotting with anti(human Fc) and anti(human kappa) antibodies (data not shown). Gels were placed onto Problott (Applied Biosystems, Foster City, Calif.) and transferred for 2 hr. at 150 milliamp. The blots were blocked for 1 hr in Blotto (5% Carnation Instant Milk in PBS), and then incubated for 1½ hr with a 1/250 dilution of either horseradish peroxidase(HRP)-conjugated goat antihuman kappa (0.4 mg/mL, Fisher Scientific, Pittsburgh, Pa.) or horseradish peroxidase-conjugated goat anti-human Fc (Jackson ImmunoResearch Lab, Code Number 109-039-098, Bar Harbor, Me.).

Immunoblotting also showed that the other species on the non-reducing gel consist of both the H and L chains. The band at 14 KDa on the reducing gel is a bacterial protein unrelated to immunoglobulin. Furthermore, N-terminal protein sequencing of the L and H chains (7 cycles each) showed that the pelB leader peptide was processed correctly to yield the mature N-termini for both the L and H chains. N-terminal protein sequencing was performed on an Applied Biosystems 477A protein sequencer.

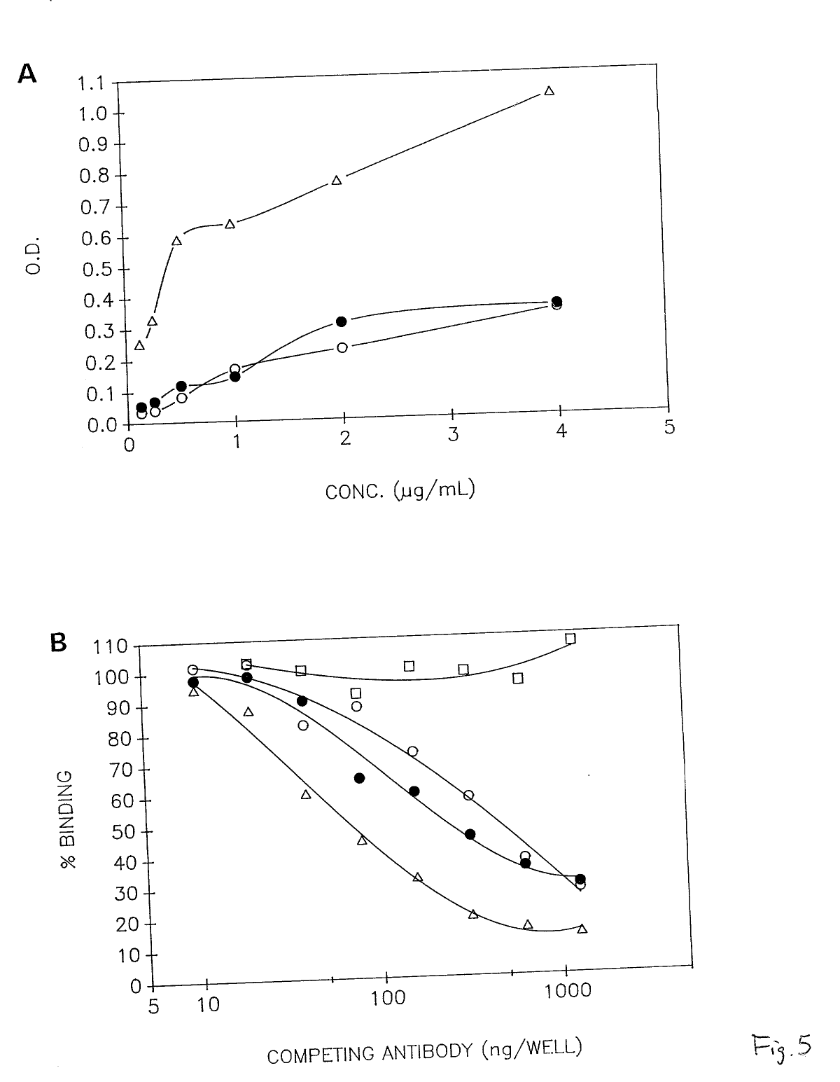

Non-denaturing size exclusion HPLC showed that the predominant species in the ch14.18ΔCH2 antibody purified from bacterial culture has an apparent mol. wt. of about 126 KDa, which agrees well with the mol. wt. of the tetrameric H2L2 (FIG. 4). FIG. 4 shows results of the non-denaturing size exclusion high pressure liquid chromatography. Chl4.18ΔCH2 antibody from E. coli was analyzed on a TSK 3000 column run in PBS, pH 7.0 (7.8×300mm) with a guard column. The running buffer was PBS, pH 7.0, at a flow rate of 0.8 mL/min. The horizontal axis is the retention time in minutes and the vertical axis is the absorbance at 214 nm. The mol wts. assigned (in KDa) were measured against Pharmacia standards (thyroglobulin, ferritin, catalase and ovalbumin). The peak eluting with an apparent mol. wt. of 126 KDa is H2L2.

5. Antigen Binding Activity.

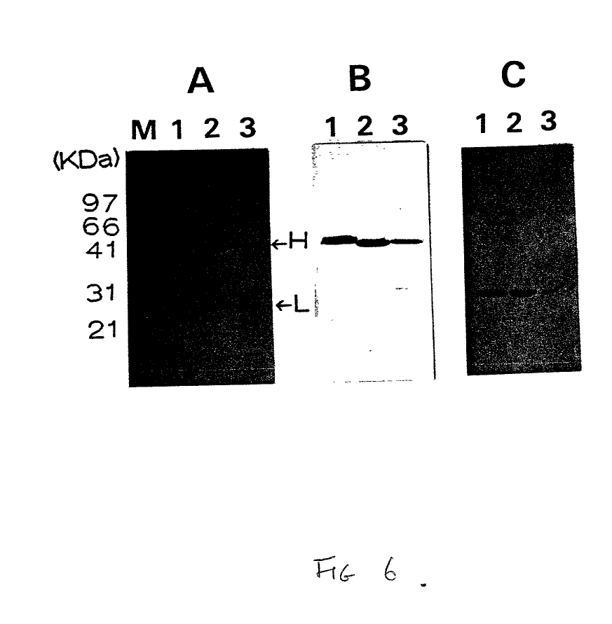

Direct antigen binding assays and competitive binding assays on GD2-coated microtiter plates (Gillies et al., 1989, J. Immunol. Methods 125:191-202) showed that the ch14.18ΔCH2 antibody from bacteria has about the equivalent antigen binding activity as the ch14.18 antibody from transfected Sp2/0 cells. FIG. 5 shows results of antigen binding assays on GD2-coated plates. The bacterial ch14.18ΔCH2 (open circles) was compared against the ch14.18 (closed circles) and ch14.18ΔCH2 (Δ) antibodies prepared from transfected Sp2/0 cells. FIG. 5A shows results of a direct antigen binding assay. Bound antibody was detected with horseradish peroxidase-conjugated anti-human K chain antibody. FIG. 5B shows results of competitive antigen binding assay. The test antibodies and tracer (a horseradish peroxidase-conjugated ch14.18 antibody, 12.5 ng/mL) were incubated at 37° C. for 2 hr. The amount of bound tracer was determined in the absence of competitor to give the 100% binding value. The anti-mucin chB72.3 antibody (Gillies et al., 1989, J. Immunol. Methods. 125:191-202)(square) was used as a negative control.

The concentration of bacterial ch14.18ΔCH2 used in the assay was based on anti(Fc) ELISA data of the material purified on the anti-human K monoclonal antibody-Sepharose column. As shown above in FIG. 2B, there are other species in addition to the H2L2, and this may account for the lower binding activity of the bacterial product. This binding activity is significantly lower than that of the ch14.18ΔCH2 from transfected Sp2/0 cells. Chl4.18ΔCH2 produced by Sp2/0 cells shows a higher rate of antigen binding than ch14.18 produced by Sp2/0 cells (Gillies et al., 1990, Hum. Antibod. Hybridomas 1:47-54). The antigen binding affinity of the ch14.18ΔCH2 from bacteria resembles that of mammalian-produced ch14.18 rather than ch14.18ΔCH2 from mammalian (Sp2/0) cells.

6. Mechanism.

Without being bound to any mechanism, a proposed mechanism for formation of a heterotetramer is that the two HL half molecules are formed and the tetramer is held together by a non-covalent trans interaction between the two CH3 domains. Results of SDS-PAGE and non-denaturing high pressure exclusion chromatography showed that the secreted product contained the dimeric HL and the tetrameric H2L2, and that inter-H chain disulphide bonds were not formed. In order to demonstrate that assembly of the H2L2 heterotetramer was not formed spontaneously as the exported polypeptides were being concentrated during purification, the concentrations of ch14.18ΔCH2 from both mammalian and bacterial sources were varied during purification, and then analyzed on SDS-PAGE. The results showed that the two half-molecules do not dissociate and associate freely during purification in the concentration range encountered in the purification steps. Thus, it is likely that a trans interaction between CH3 domains takes place while the immunoglobulin is inside the bacteria, possibly in the periplasmic space.

7. Construction of Bacterial Expression Vector pKKx-pelB 14.18

The expression vector pKKx-pelB 14.18 is identical to pKKx-pelB 14.18ΔCH2 except that the complete H-chain cDNA replaces the ACH2 H-chain cDNA.

Poly A⁺ enriched mRNA was prepared from a transfected Sp2/0 cell line which produces the ch14.18 antibody (Gillies et al., 1989, J. Immunol. Methods 125:191-202). First strand cDNA synthesis and PCR were performed as described in Example 1 above.

In the 14.18 H-chain cDNA, there are two restriction sites flanking the CH2 domain: a NarI site in the CH1 domain and an XmaI site in the CH3 domain. The H-chain cDNA clone containing the correct sequence between these two sites was used for reconstruction. The NarI-XmaI fragment of this H-chain CDNA clone was isolated and used to replace the NarI-XmaI fragment in the expression vector pKKx-pelB 14.18ΔCH2 to give pKKx-pelB 14.18, which contains DNA encoding the complete H chain and the L chain.

8. Expression of pelB-ch14.18

The expression of pKKx-pelB ch14.18 in JM105 was carried out essentially as described in Example 2, except that LB-amp was used instead of M9-amp in order to increase the level of expression of the H and L chain genes. As was found with pKKx-pelB ch14.18ΔCH2, before, the trc promoter in pKKx-pelB ch14.18 was not fully repressed and IPTG induction did not significantly improve the expression level. The expression level of ch14.18 was 200 ng/mL, as determined by anti(H+L) and 120 ng/mL by anti(Fc) ELISA, indicating that there is free L chain or L chain dimer secreted into the media.

9. Purification of ch14.18 Antibody from E. coli

16 L of clarified LB-amp culture medium was passed through an Amicon hollow fibre cartridge (HIMP01-43, Amicon, Danvers, Mass.) to remove any residual bacterial debris. Sodium azide was added to a final concentration of 0.02% and sodium hydroxide added to pH 7.0. A column packed with 5 mL of Prosep A (Bioprocessing Ltd., Durham, England) was equilibrated in a buffer containing 2.75 mM sodium citrate, mM sodium phosphate and 150 mM NaCl at pH 8.0. The sample was loaded at 500 mL/hr at 4° C. The column was washed with a buffer containing 2.75 mM sodium citrate, 194 mM sodium phosphate and 500 mM NaCl at pH 8.0, and then eluted with a buffer containing 61 mM sodium citrate, 71 mM sodium phosphate and 150 mM NaCl at pH 4.0. After the Prosep A column, the sample was further purified by a 5-mL murine anti-human kappa Sepharose 4B column, as described in Example 2, and concentrated in an Amicon stirred cell with a Diaflo ultrafiltration membrane YM5 to 0.38 mg/mL, as determined by anti(Fc) ELISA.

10. Electrophoretic Analysis and Immunoblotting of ch14.18 Antibody from E. coli

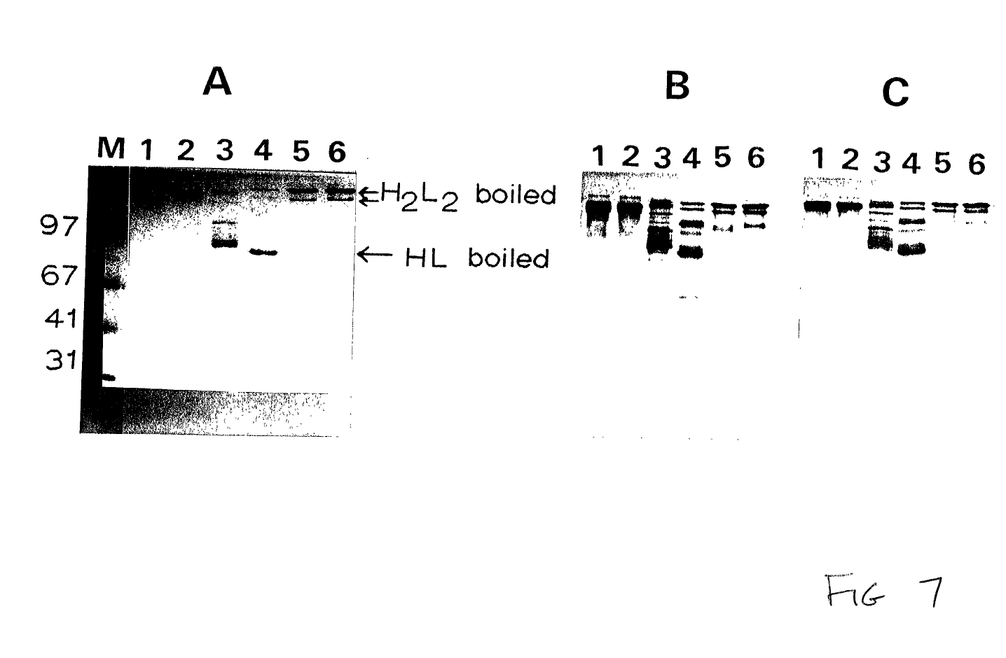

FIGS. 6 and 7 show the electrophoretic analysis and immunoblotting of the ch14.18 antibody purified from culture media of E. coli. The ch14.18 antibody purified from spent culture of transfected Sp2/0 cells was used for comparison. For a proper comparison of the E. coli and mammalian cell-produced antibodies, the mammalian ch14.18 was treated with N-glycanase (Genzyme) to remove the carbohydrates since ch14.18 from transfected Sp2/0 cells is N-glycosylated (in the CH2 domain) and the ch14.18 from E. coli is not.

In FIG. 6, samples were analyzed in a 10% SDS polyacrylamide gel after reduction with 2-mercaptoethanol. In FIG. 6A, the gel was stained with Coomassie blue. Lane 1 shows ch14.18 from transfected Sp2/0 cells; lane 2, ch14.18 from transfected Sp2/0 cells treated with N-glycanase; lane 3, ch14.18 from E. coli. The positions of the H and L chains are as indicated. There is a slight shift in mobility of the H chain when the mammalian ch14.18 was treated with N-glycanase, resulting in a deglycosylated H chain which comigrates with the H chain of bacterial ch14.18. The identities of the bands assigned H and L were confirmed by immunoblotting (for details, see Example 4) with HRP-conjugated anti(human Fc) antibody (FIG. 6B) and with HRP-conjugated anti(human kappa) antibody (FIG. 6C).

In FIG. 7, samples (boiled or not boiled) were run on a 10% SDS polyacrylamide gel under non-reducing conditions. FIG. 7A is a Coomassie staining of the gel, and FIGS. 7B and 7C are immunoblotting with HRP-conjugated anti(human Fc) and anti(human kappa) antibodies respectively. In FIGS. 7A, B and C, lanes 1 and 5 show ch14.18 from transfected Sp2/0 cells treated with N-glycanase; lanes 2 and 6 show ch14.18 from transfected Sp2/O cells; lanes 3 and 4 show ch14.18 from E. coli. Lanes 1-3 show non-boiled samples and lanes 4-6 are boiled samples. The many extra bands in the nonboiled bacterial ch14.18 of lane 3 are probably a result of nonspecific interactions among the different protein species. If the bacterial sample is boiled before loading, the HL half molecule became the dominant species as in lane 4. In lane 4, there are bands that comigrate with the mammalian ch14.18 in lanes 5 and 6 (indicated as “H2L2 boiled”). Since mammalian ch14.18 contains inter-H chain disulphide bonds, the mammalian produced immunoglobulin apparently remains in the sample as a tetrameric molecule after boiling. The fact that the bacterial ch14.18 gave the same pattern of bands after boiling suggests that inter-H chain disulphide bonds are also formed in E. coli.

11. Analysis of ch14.18 Antibody from E. coli by Protein Sequencing and HPLC

N-terminal protein sequencing (see Example 4) of the L and H chains (10 cycles each) showed that the pelB leader peptide was processed correctly to yield the mature Nterminus for both the L and H chains.

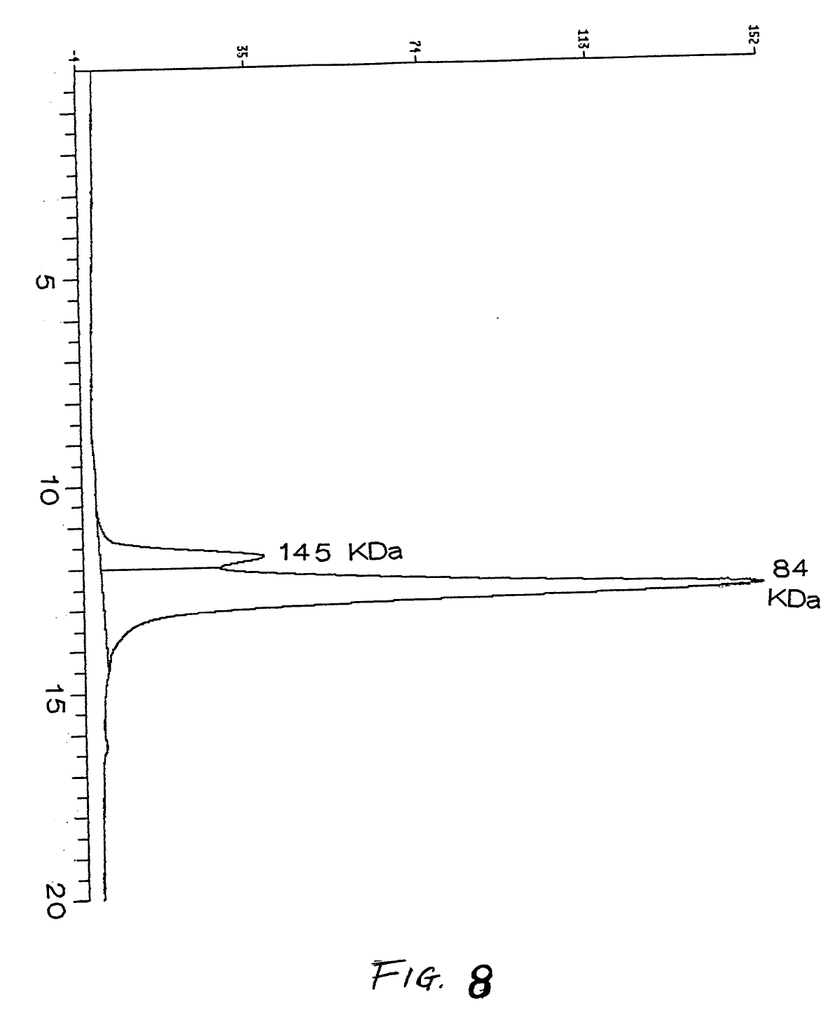

Non-denaturing size exclusion HPLC was performed as in Example 4. The results showed that the ch14.18 purified from bacterial culture contains a peak with an apparent molecular weight of approximately 145 KDa, which agrees well with the mol. wt. of the aglycosylated tetrameric H2L2 (FIG. 8). The major peak in the HPLC has an apparent mol. wt. of approximately 84 KDa, which corresponds to the HL halfmolecule.

12. Antigen-binding Activity of ch14.18 from E. coli

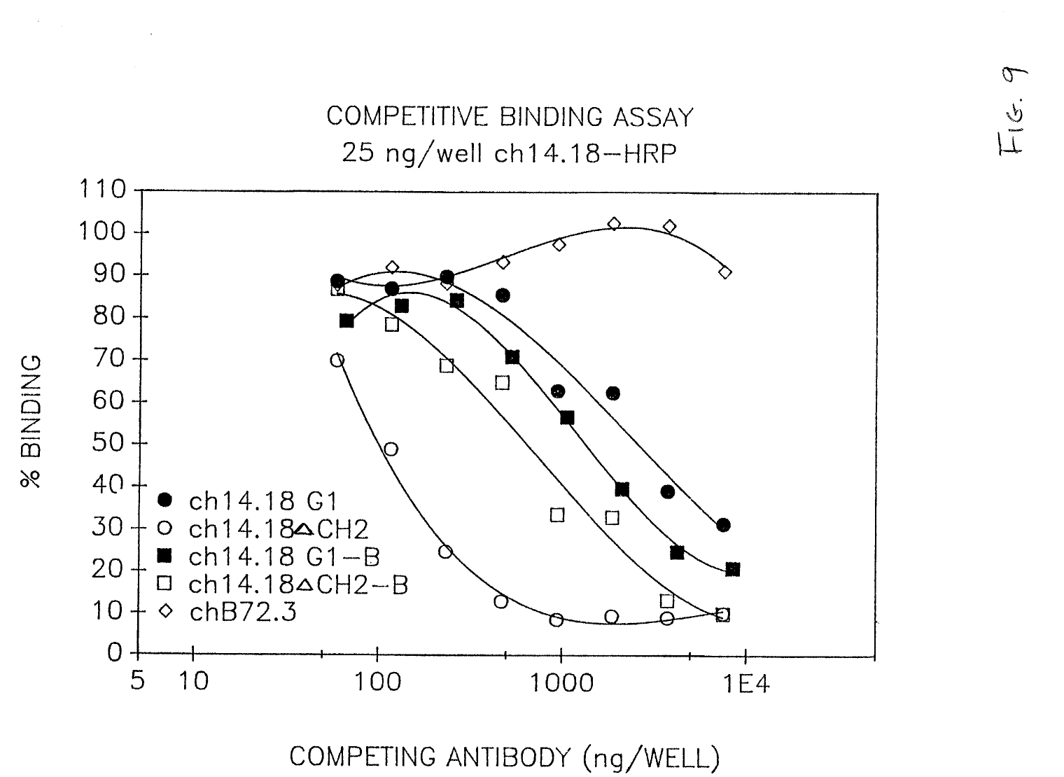

Direct antigen binding assays and competitive binding assays on GD2-coated microtiter plates were performed as described in Example 5. Direct antigen binding showed that the ch14.18 antibody from bacteria retains GD2 binding activity. Results of the competitive binding assay are shown in FIG. 9. The bacterial ch14.18 (solid squares) competes at least as effectively as the ch14.18 prepared from Sp2/0 cells (solid circles). Also included in the assay were the bacterial ch14.18ΔCH2 (open squares) and ch14.18ΔCH2 from transfected Sp2/0 cells (open circles), which act as positive controls, and the chB72.3 antibody as a negative control.

13. Immunoglobulin Conjugates

E. coli produced immunoglobulin conjugates may be made as described above, except that the protein to be conjugated to the Ig molecule can be fused at the DNA level to the H encoding DNA, according to conventional genetic engineering techniques. The resultant fusion protein will include an independently biologically functional polypeptide bonded to the C-terminus of the CH3 domain, e.g., a lymphokine, cytokine, or cell toxin. The resultant fused protein will be expressed and exported from E. coli, as the unfused Ig molecule is.

What is claimed is:

1. A method of producing recombinant heterotetrameric immunoglobulin from a procaryotic organism, said method comprising
proviidng a procaryotic organism that has been transformed with DNA encoding the heavy and light chains of an immunoglobulin having a binding site for immunologically binding a preselected antigen and an amino acid sequence which signals the export of said immunoglobulin from the cytoplasm of said organism, said DNA being operationally associated with a promoter recognizable by RNA polymerase endogenous to said organism, and
culturing said transformed procaryote for a time and under conditions sufficient to allow said organism to export said immunoglobulin from the cytoplasm of said organism, wherein said exported heterotetramaeric immunoglobulin retains its native conformation and binding specificity for said preselected antigen.

2. The method claim 1 wherein said procaryotic organism is a gram negative bacterium.

3. The of claim 2 wherein said gram negative bacteria comprise E. coli.

4. The method of claim 3 wherein said export sequence comprises one of the E. coli pelB, ompA, or phoA signal sequences.

5. The method of claim 3 wherein said export sequence comprises pelB and said immunoglobulin is secreted into the culture medium surrounding said cultured bacteria.

6. The method of claim 4 wherein said heavy chain encoding DNA contains a deletion of DNA encoding the immunoglobulin CH2 domain.

7. The method of claim 1 wherein said DNA encoding heavy chain encodes full length constant region.

8. A DNA encoding a recombinant heterotetrameric immunoglobulin comprising a heavy chain and a light chain and an amino acid sequence which directs export of said immunoglobulin out of the cytoplasm of a procaryotic organism, said DNA being operationally associated with a DNA defining a promoter recognizable by an RNA polymerase endogenous to said organism wherein, upon translation within said organism of mRNA transcribed from said DNA, a tetrameric immunoglobulin comprising a binding site for a preselected antigen is exported in its native antigenbinding conformation from the cytoplasm of said organism.

9. The DNA of claim 8 wherein said promoter is recognizable by an RNA polymerase recognized by a gram negative bacterium.

10. The DNA of claim 9 wherein said gram negative bacterium comprises E. coli.

11. The DNA of claim 10 wherein said export sequence comprises one of the E.coli pelB, ompA, or phoA signal sequences.

12. The DNA of claim 10 wherein said export sequence comprises pelB.

13. The DNA of claim 8 wherein said heavy chain comprises a deletion of acids comprising the CH2 domain.

14. The DNA of claim 8 wherein said DNA encoding heavy chain encodes full length constant region.

15. The DNA of claim 8 wherein said heavy chain comprises a CH3 domain.
